(19) United States
(12) Patent Application Publication
(10) Pub. No.: US 2011/0021435 A1
(43) Pub. Date: Jan. 27, 2011

(54) COMPOSITION FOR PREVENTING OR TREATING BRAIN DISEASES

(75) Inventors: Heejae Lee, Chuncheon (KR); Jong-Seon Byun, Chuncheon (KR); Kyungyoung Lee, Chuncheon (KR); Sangjung Baik, Chuncheon (KR); Dahlkyun Oh, Chuncheon (KR)

Correspondence Address: Edwards Angell Palmer & Dodge LLP, P.O. Box 55874, Boston, MA, 02205, US

(73) Assignee: REGERON, INC., Chuncheon (KR)

(21) Appl. No.: 12/843,690

(22) Filed: Jul. 26, 2010

Related U.S. Application Data
(63) Continuation-in-part of application No. PCT/KR2009/000364, filed on Jan. 23, 2009.

(30) Foreign Application Priority Data
Jan. 25, 2008 (KR) 10-2008-0008207

(51) Int. Cl. A61K 38/17; A61P 25/24; A61P 25/28

(52) U.S. Cl. 514/17.6; 514/17.7

(57) ABSTRACT

The present invention relates to a composition for preventing or treating neurological diseases, particularly brain diseases and improving cognitive functions by inhibiting apoptosis of neuronal cells and/or promoting generation of neuronal cells. The present invention provide a composition for preventing or treating a neurological disease, particularly brain disease, and a composition for improving a cognitive function, which comprises stanniocalcin 2 as an active ingredient.

### CROSS-REFERENCE TO RELATED APPLICATION

This is a continuation-in-part of International Application PCT/KR2009/000364, with an international filing date of Jan. 23, 2009, which claims the benefit of Korean Application No. 10-2008-0008207 filed Jan. 25, 2008, the entire contents of which are incorporated herein by reference.

### BACKGROUND OF THE INVENTION

1. Field of the Invention

The present invention relates to compositions and methods for preventing or treating a neurological disease and a composition for improving a cognitive function, and more specifically, the composition for preventing or treating a neurological disease (particularly brain disease), and the composition for improving the cognitive function which comprises stanniocalcin 2 as an active ingredient.

2. Description of the Related Art

A large number of factors are known to be involved in the onset of neuronal diseases. Examples of such factors include downregulated neurogenesis and microglial activation. Accordingly, prior arts have reported findings relevant to potential treatments of neuronal diseases based on the induction of neurogenesis and/or suppression of the microglial activation.

In relation to this approach, induction of neurogenesis have been implicated in treatment of various diseases; 1) epilepsy or seizure (Hattiangady et al. Neurobiology of disease, 17 (3): 473-490 (2004), Cho et al. J Korean neurological association, 23: 503-509 (2005)), 2) Parkinson's disease (He et al. J. toxicologic pathology, 22 (2): 101-108 (2009); Yoshimi et al. Annals of neurology, 58 (1): 31-40 (2005)), 3) Depression or Cortical Spreading Depression (Sahay, et al. Prog Brain Res., 163: 697-722 (2007); Malberg, et al. J Neurosci., 20: 9104-9110 (2000)), 4) Schizophrenia (Reif, et al. Mol Psychiatry, 11: 514-522 (2006)), 5) Alzheimer's disease (Galyan, et al. CNS Neurol Disord Drug Targets, 6: 303-310 (2007); Tatebayashi, et al. Acta Neuropathol., 105: 225-232 (2003)), and 6) Frontotemporal lobar degeneration (FTLD; Pick's disease) (Armstrong, et al., Neuropathol., 20: 170-175 (2001)).

Microglia is a type of glial cells that are resident macrophages of the brain and spinal cord, and thus act as the first and main form of active immune defense in the central nervous system (CNS). By undergoing a variety of structural changes based on their locations and given roles, they have diversified functions which include constant excavation of the CNS for damaged neurons, destroying infectious organisms via phagocytosis, and secretion of anti-inflammatory cytokines, and recruitment of neurons to the damaged area. Without microglial cells, regrowth and remapping would be considerably slower.

However, in some cases of neural inflammation (neuroinflammation) or injury, microglia release a variety of inflammatory cytokines and/or cytotoxic substances, hence can injure neurons resulting in neurodegenerative symptoms such as plaque formation, thereby contribute to and expand the neurodestructive effects worsening the disease processes (Streita et al. Trends in Neurosciences, 29 (9): 506-510 (2006)). As a result, responses to those neural inflammation and injuries can result in a large scale neural damage as the microglia ravage the brain in an attempt to destroy the invading infection and/or clear the damaged neuronal cells or tissues (Gehrmann. Et al. Brain Research Reviews, 20 (3): 269-287 (1995)).

Accordingly, the activation of microglia has been shown to be involved in several neuronal disorders; 1) epilepsy and/or seizure (Wirenfeldt et al. Neurobiology of disease, 34 (3): 432-44 (2009); Taniwaki et al. Neuroscience research: the official journal of the Japan Neuroscience Society, 24/26 (20): S80 (1996)), 2) Parkinson's disease (Long-Smith, et al. Prog Neurobiol., 89: 277-287 (2009)), 3) Alzheimer and Parkinson's diseases (Laskowitz, et al. Exp Neurol., 167: 74-85 (2001); Itagaki et al. Advances in behavioral biology, 38 (A): 381 (1993)). In addition, suppression of microglial activation has been shown to be linked to protection of neuronal cells (Li, et al. J Neurosci Res., 66: 163-170 (2001)).

Based on these prior arts, one embodiment of this invention presents use of STC2 for suppression of phenotypes and/or improvement of functions related to neuronal disorders in non-human in vivo models representing disorders caused by neuronal loss and/or neurogenesis down-regulation on one hand, and also disorders of neuroinflammmation and/or neurodegeneration resulting from microglial activation on the other. Other embodiments include use of STC2 for improvement of cognitive functions and/or behavioral performances related to the same.

Kainic acid (KA) is an excitotary cytotoxin capable of eliciting microglial activation (Taniwaki et al. Neuroscience Letters, 217 (1): 29-32 (1996)). When administered intracerebroventricularly (i.c.v.) in mice, KA induces markedly concentrated morphological damage and cell death in the hippocampal CA3 pyramidal neurons resulting in learning and memory impairment (Lee et al., Brain Res Bull., 61 (1): 99-107 (2003)).

Prior arts have shown that kainic acid treatment resulted in in-vivo models with neuronal diseases, such as epilepsy, seizures (Urino et al., Neurologia medico-chirurgica, 50 (5): 355-360 (2010)), Parkinson's disease (Foster & Levine Chemistry and biology of pteridines and folates, 2002, Chap. 8, pp. 393-398; Tetrahydrobiopterin (BH₄)-mediated neuronal death following intrastriatal kainic acid: Implications for Parkinson's Disease.), and Cognitive Dysfunction (Srivastava et al. Neurochemical research, 33 (7): 1169-1177 (2008)).

It is from this perspective that we have injected KA intracerebroventricularly (i.c.v.) into mouse brain to prove the efficacy of Stanniocalcin 2 (STC2) in treating the neuronal disorders.

Stanniocalcin 2 (STC2) is a homodimeric glycoprotein like its paralog stanniocalcin 1 (STC1) (Luo et al. Endocrinology, 146 (1): 469-476 (2005)). STC2 share dissimilarities and similarities with STC1 in biological and physiological properties as described below.

Unlike STC1, the level of serum Ca2+ and PO4 were unchanged in STC2-overexpressing transgenic mice, although STC-1 could regulate intra- and extracellular Ca2+ in mammals (Gagliardi et al., 2005, v288 no. 1=v51 no. 1, pp. 92-105). In contrast to STC1, STC2 is not highly expressed during development but exhibits overlapping expression with STC1 in adult mice, with heart and skeletal muscle exhibiting the highest steady-state levels of STC2 mRNA (ibid.). STC2 is secreted as phosphoproteins and is phosphorylated in vitro by casein kinase II (CK2), while STC1 is phosphorylated in vitro by protein kinase C (PKC) exclusively on serine residues (Jellinek et al. Biochemical journal, 350 (2): 453-461 (2000)). STC2 is known to be located in Golgi and endoplasmic reticulum, while STC1 is mainly present in inner mitochondria (mitoplasts) (Ito et al. Mol Cell Biol, 24: 9456-9469 (2004); McCudden et al. 277: 45249-45258 (2002)). STC2-transfected CHO cells inhibited the phosphate uptake of a kidney cell line, whereas STC1 showed no inhibitory effects (Ishibashi et al. Biochemical and biophysical research communications, 250 (2): 252-258 (1998)). The function of STC2 seems to be opposite to that of STC1 on Na-phosphate cotransporter (ibid.). It has been also demonstrated that they have different profiles in cancer cells: expression of STC1 was induced by BRCA1, a tumor suppressor gene that has an important role in breast and ovarian cancer. On the other hand, the expression of STC2 is induced by estrogen (Jellinek et al. Endocr Relat Cancer, 10 (3): 359-73 (2003)). Furthermore, the antibodies of STC1 and STC2 do not recognize the epitope of the other stannicalcin paralog (McCudden et al. 277: 45249-45258 (2002); Ito et al. Mol Cell Biol, 24: 9456-9469 (2004)). Moreover, addition of excess STC2 could not displace STC1-fusion protein bound to STC1 receptor (ibid.).

Similar to STC1, STC2 can act as a potent growth inhibitor and reduce intramembranous and endochondral bone development and skeletal muscle growth (Gagliardi et al. Am J Physiol Endocrinol Metab., v.288 no.1=v.51 no.1, pp. 92-105 (2005)). Such growth-suppressive properties of human stanniocalcin-2 in transgenic mice were shown to be exerted independently from growth hormone and IGFs (Gagliardi et al. Am J Physiol Endocrinol Metab., 288 (1): E92-105 (2005)). Northern analysis revealed that mammalian STC2, like STC1, was expressed in a wide variety of tissues (Shin et al. Comparative biochemistry and physiology. Part A, Molecular & integrative physiology, 153 (1): 24-29 (2009)). STC2, like STC1, were found to be expressed in multiple tissues as paracrine regulators (ibid.).

STC2 shares amino acid sequence identity to STC1 by less than 35% (Ishibashi et al., Biochemical and biophysical research communications, 1998, v250 no.2, Ishibashi et al. Am J Physiol Renal Physiol., 282 (3): F367-75 (2002); Chang et al. Molecular and cellular endocrinology, 141 (1/2): 95-99 (1998)). However, Blast analysis results indicate that the nucleotide sequence of human STC2 has no hits with significant matching with those of STC1 regardless of its species or tissue origin. Most importantly, in contrast to STC1, the predicted amino acid sequence of STC2 contains a cluster of histidine residues in the C-terminal portion of the protein implying additional functions in relation to metal binding (Shin et al. Comparative biochemistry and physiology. Part A, Molecular & integrative physiology, 153 (1): 24-29 (2009)). Unlike STC1, both the N- and the C-terminal fragments of STC2 were hypocalcemic, causing 18 and 12% reduction in plasma calcium level in eel (Verbost et al., General and comparative endocrinology, 98 (2): 185-192 (1995)) in that the hypocalcemic activity of the C-terminal fragment was suggested to be due to its effect on calcium influx, while the N-terminal fragment appears to function in a different manner.

STC2 and STC1 in neural cell activities also share dissimilarities and similarities: STC2 expression was activated in neuronal cells by oxidative stress and hypoxia via mechanisms involving UPR (unfolded protein response), but not by several other cellular stresses unrelated to the UPR, while the STC1 expression was upregulated by hypoxia in a different manner (Ito et al. Molecular and cellular biology, 24 (21): 9456-9469 (2004)). Earlier studies identified a high level of constitutive contents of STC1 mammalian brain neurons (Serlachius et al. Peptides., 25 (10): 1657-1662 (2004)), and the expression of STC1 being related to terminal differentiation of neural cells (ibid. and Koide et al., Rinsho Byori., 54 (3): 213-220 (2006)). It was also suggested that the altered expression of STC1 contributes to the protection of cerebral neurons against hypoxic/ischemic damage (Zhang et al. Proc Nat/Aced Sci USA., 28; 97 (7): 3637-42 (2000)). STC1 may act as a regulator of calcium homeostasis in terminally differentiated brain neurons (Zhang et al., The American journal of pathology, v.153 no.2, 1998, pp. 439-445). Both STC2 and STC1 were suggested to be pro-survival factors for the endurance of terminally differentiated cells such as neurons and adipocytes (Joensuu et al. Cancer letters, 265 (1): 76-83 (2008)). A study using cDNA microarray technology demonstrated that STC2 gene is upregulated by responding to β-amyloid in human neuroblastoma cells (Kim et al. Experimental & molecular medicine, 35 (5): 403-411 (2003)). In human brain microvascular endothelial cells, stanniocalcin-1 (STC1) was also shown to be upregulated by β-amyloid treatment in a time and dose-dependent manner (Li et al. Biochemical and biophysical research communications, 376 (2): 399-403 (2008)). According to the claims made in WO0108697, stanniocalcin 2 and its biologically functional derivatives and fragments are useful in the diagnosis and treatment of type II diabetes and chronic conditions associated with diabetes (ibid.). The use of STC1 has been disclosed for treating hypocalcemia and osteoporosis (JP10509036T), detecting leukemia (JP2000002709A).

Patent applications claiming the use of STC1 for treating neuronal diseases or protecting damaged neuronal cells were previously disclosed (WO0130969 and the families, US20020042372 and US20040198658). However it should be emphasized that in these applications, the neuroprotective functions of STC1 have been mainly implicated for disorders related to hypoxic stress, such as cerebral infarction, ischemia, stroke or injuries due to attack or thromboembolism, or calcium mediated diseases, but not seizures/epilepsy, Alzheimer's disease, Parkinson's disease, or cognitive/behavioral deficits which are the targeted disorders that this invention attempts to treat and prevent using STC2 through induction of neurogenesis and/or suppression of the microglial activation.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present invention relates to the development of novel therapeutics and methods for preventing or treating brain diseases and improving cognitive functions by suppressing microglial activation and promoting generation of neuronal cells. The present inventors have made intensive researches to develop novel therapeutics for preventing or treating brain diseases and improving cognitive functions by inhibiting apoptosis of neuronal cells and promoting generation of neuronal cells. As a result, we have discovered that stanniocalcin 2 has the activities described above for neuronal cells.

Accordingly, it is one object of this invention to provide a composition for preventing or treating a brain disease, which comprises stanniocalcin 2 as an active ingredient.

It is another object of this invention to provide a composition for improving a cognitive function, which comprises stanniocalcin 2 as an active ingredient.

It is still another object of this invention to provide a method for preventing or treating a brain disease.

It is further object of this invention to provide a method for improving a cognitive function.

Other objects and advantages of the present invention will become apparent from the detailed description to follow taken in conjugation with the appended claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 represents that stanniocalcin 2 (STC2) prevents neuronal death in cornu Ammonis 3 (CA3) of mouse hippocampus. To compare KA alone with KA and STC2, panel A and C are treated with kainic acid (KA) alone, and panel B and D are treated with both KA and STC2. In panel A, each CA1, CA2 and CA3 indicates cornu Ammonis (CA) field 1, field 2 and field 3 of hippocampus, and DG indicates dentate gyrus (DG). In panel C, three block arrows represent an apoptotic region of neuronal cell. In panel B and D, it was demonstrated that STC2 enables to inhibit neuronal death.

FIG. 2 represents genome of postmitotic neurons stained with bromodeoxyuridine (BrdU) using immunohistochemistry to examine STC2 effects on neuron proliferation in subgranular zone (SGZ) located in mouse hippocampus. Black arrows indicate BrdU-immunopositive cells. It could be demonstrated that BrdU-immunopositive cells in panel B and D (STC2) are increased in SGZ compared to panel A and C (control).

FIG. 3 is a comparative graph relatively quantifying experimental results of FIG. 2. Control is a group without STC2, and STC2 is a group with STC2. It could be appreciated that BrdU-immunopositive cells in SGZ are significantly enhanced in STC2-treated group compared with control.

FIG. 4 shows SDS-PAGE on precipitates centrifuged after Top10F′ cells transformed with pUC-narK Met-hSTC2 are homogenized, indicating 33 kDa band corresponding to hSTC2.

FIG. 5 is SDS-PAGE of purified hSTC2, indicating 33 kDa band of purified hSTC2.

FIG. 6 is SDS-PAGE of purified hSTC2, indicating 33 kDa band of purified hSTC2. Lane 1 is protein size marker; and lane 2 is purified hSTC2.

FIG. 7 represents Y-maze behavior in mouse, demonstrating that the score of place memory in hSTC2-treated group is significantly increased compared to KA alone-treated group (p<0.05).

FIG. 8 is a water finding test in mouse and drinking latency in hSTC2-treated group is significantly reduced compared to KA alone-treated group, meaning excellent learning memory (p<0.05).

FIG. 9 is a forced swim test in mouse and immobile time in hSTC2-treated group is significantly reduced compared to PB-treated group, representing effective efficacy on improvement of depression-related behavior (p<0.05).

FIG. 10 represent the extent of brain impair in hSTC2-treated group is significantly reduced in mouse transient focal ischemic model compared to that in PB-treated group, demonstrating effective reduction in volume of cerebral infraction and neurological deficit (p<0.05).

### DETAILED DESCRIPTION OF THIS INVENTION

In one aspect of this invention, there is provided a composition for preventing or treating a brain disease, which comprises stanniocalcin 2 as an active ingredient.

In another aspect of this invention, there is provided a composition for improving a cognitive function, which comprises stanniocalcin 2 as an active ingredient.

In still another aspect of this invention, there is provided a method for preventing or treating a brain disease, which comprises administering to a subject a pharmaceutical composition comprising stanniocalcin 2 as an active ingredient.

In further aspect of this invention, there is provided a method for improving a cognitive function, which comprises administering to a subject a pharmaceutical composition comprising stanniocalcin 2 as an active ingredient.

The most striking feature of the present invention resides on our novel findings in which STC2 inhibits neuronal death and promotes generation of neuronal cells.

The composition of this invention comprising STC2 is very effective in preventing or treating a variety of neurologic diseases, inter alia, brain diseases. The therapeutic effects of the present composition are ascribed to its neuroprotective actions. The term used herein “neuronal cell” refers to central nervous system, brain, brainstem, spinal cord, neuron having a structure connecting central nervous system and peripheral nervous system, and neuronal supporting cell, Glia and Schwann cell. As used herein, the term “protective activity for neuronal cell” refers to the effect of reducing or ameliorating neurologic insult, and protecting or reviving neuronal cell that has suffered neurologic insult. In addition, the term “neurologic insult” used herein means any damage to neuronal cell or tissue resulting from various causes such as metabolic, toxic, neurotoxic and chemical causes.

A Practical example of disease or disorder applicable to the composition of the present invention includes, but not limited to, a neurodegenerative disease, an ischemia-reperfusion injury and a mental disorder. More specifically, the composition of the present invention may be utilized as a composition for preventing or treating a neurodegenerative disease such as Alzheimer's disease, Huntington's disease, Parkinson's disease and amyotrophic lateral sclerosis (ALS); ischemia or reperfusion injury such as stroke (particularly, ischemic stroke); and mental disorder such as schizophrenia, depression, manic depression and post traumatic stress disorder.

As shown in Examples below, stanniocalcin 2 (STC2) of the present invention may remarkably inhibit a cell death via a neuronal apoptosis. For example, STC2 may significantly inhibit neuronal death by kainic acid as a neurotoxic substance which induces a neuronal apoptosis.

Stanniocalcin 2 (STC2) of the present invention may strikingly improve a cognitive function. Preferably, STC2 of the present invention has a superior activity for improving or preventing impairment of cognitive function caused by the above-described neurological diseases. In addition, STC2 of the present invention has an excellent efficacy on improvement of cognitive function in the normal person.

Meanwhile, hippocampus of the brain is the most important region in the formation and storage of memory. Hippocampus is a neuron-dense region in which new neuronal cells is actively produced and is responsible for learning and memory function via reciprocal electrical stimulation. STC2 of the present invention promoted neurogenesis, particular in subgranular zone (SGZ) beneath granular cell layer (GCL) of dentate gyrus (DG) inside hippocampus. Given that imipramine as a representative antidepressant has no effect on treatment of depression where neurogenesis in hippocampus is not generated, neurogenesis in GCL of hippocampal DG may be associated with improvement of stress. As another antidepressant paroxetine is known to promote neurogenesis in GCL of hippocampal DG, it is preferable that stanniocalcin 2 is utilized as a therapeutic agent against depression.

According to a preferable embodiment, STC2 of the present invention has an activity for improvement of cognitive function, for example including improvement of learning ability and/or memory.

The term “prevention” used herein refers to inhibiting the generation of disorders or diseases in animal who are not diagnosed to have but are susceptible to such disorders or diseases. As used herein, the term “treatment” refers to (a) inhibiting the development of disorders or diseases; or (b) ameliorating or (c) removing the disorders or diseases.

The term “stanniocalcin 2” used herein refers to human stanniocalcin 2 unless otherwise indicated, and preferably an amino acid sequence of SEQ ID NO:1.

According to a preferable embodiment, the composition of this invention is pharmaceutical composition or a food composition.

The pharmaceutical composition of this invention includes (a) a therapeutically effective amount of stanniocalcin 2; and (b) a pharmaceutically acceptable carrier.

In the pharmaceutical compositions of this invention, the pharmaceutically acceptable carrier may be conventional one for formulation, including carbohydrates (e.g., lactose, amylase, dextrose, sucrose, sorbitol, mannitol, starch, cellulose), acacia rubber, calcium phosphate, alginate, gelatin, calcium silicate, fine crystallite cellulose, polyvinylpyrrolidone, cellulose, water, syrups, salt solution, alcohol, Arabian rubber, vegetable oil (e.g., corn oil, cotton seed oil, soybean oil, olive oil and coconut oil), poly(ethylene glycol), methyl cellulose, methylhydroxy benzoate, propylhydroxy benzoate, talc, magnesium stearate, and mineral oils, but not limited to. The pharmaceutical composition according to the present invention may further include a lubricant, a humectant, a sweetener, a flavoring agent, an emulsifier, a suspending agent, and a preservative, but not limited to. Details of suitable pharmaceutically acceptable carriers and formulations can be found in Remington's Pharmaceutical Sciences (19th ed., 1995), which is incorporated herein by reference.

The pharmaceutical composition according to the present invention may be administered via the oral or parenterally. When the pharmaceutical composition of the present invention is administered parenterally, it can be done by intravenous, subcutaneous, intramuscular and intracerebroventricular administration.

A suitable dose of the pharmaceutical composition of the present invention may vary depending on pharmaceutical formulation methods, administration methods, the patient's age, body weight, sex, severity of diseases, diet, administration time, administration route, an excretion rate and sensitivity for a used pharmaceutical composition. Physicians with average skill may easily determine and diagnose dosage level of medicine effective for treating or preventing target disorders or diseases. Preferably, the pharmaceutical composition of the present invention is administered with a daily dose of 0.0001-100 mg/kg (body weight).

According to the conventional techniques known to those skilled in the art, the pharmaceutical composition may be formulated with pharmaceutically acceptable carrier and/or vehicle as described above, finally providing several forms including a unit dose form and a multi-dose form. Formulation may be oil or aqueous media, resuspension or emulsion, extract, powder, granule, tablet and capsule and further comprise dispersant or stabilizer.

The composition of the present invention may be provided as a food composition, particularly a functional food composition. The functional food composition of the present invention may be formulated in a wide variety of forms, for example, including proteins, carbohydrates, fatty acids, nutrients, seasoning agents and flavoring agents. As described above, an example of carbohydrate may include monosaccharides (e.g., glucose, fructose, etc.); disaccharides (e.g., maltose, sucrose, etc.); oligosaccharides; polysaccharides (e.g., common sugars including dextrin, cyclodextrin, etc.); and sugar alcohols (e.g., xylitol, sorbitol, erythritol, etc.). The formulation of flavoring agent may use natural flavoring agents (e.g., thaumatin, stevia extract (e.g., rebaudioside A, glycyrrhizin), etc.) and synthetic flavoring agents (e.g., saccharine, aspartame, etc.). In the formulation of drinking agent, it may further include citric acid, liquid fructose, sweet, glucose, acetic acid, malic acid, fruit syrup, eucommia bark extract, jujube extract and glycyrrhiza extract. Considering easy accessibility of food, the food composition herein is very useful in prevention or treatment of brain disorders or diseases, or improvement of cognitive function.

The features and advantages of the present invention will be summarized as follows:

(i) The present invention provide a composition for preventing or treating a neurological disease, particularly brain disease, and a composition for improving a cognitive function, which comprises stanniocalcin 2 as an active ingredient.

(ii) Stanniocalcin 2 as the active ingredient of the present invention has a superior activity for inhibiting neuronal apoptosis, and interestingly promoting neurogenesis.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Materials and Methods

### 1. Stanniocalcin 2 (STC2) Preparation

Genomic DNA was extracted from HEF (Human embryonic fibroblast) and used as template after cutting with BamHI (Takara, Japan). PCR was carried out to obtain four DNA fragments for exon encoding stanniocalcin 2. To ligate exon DNA fragments, primers were designed for base pairing between 19 bases in a linking region, and PrimeSTAR™ HS DNA polymerase (Takara, Japan) was used in all PCR reactions. To amplify exon 1, 2, 3 and 4 of stanniocalcin 2, the first PCR method is as follows: (a) genomic DNA cut with BamHI (Takara, Japan) was commonly used as a template; and (b) PCR cycle (98° C. 10 sec; 55° C. 5 sec; and 72° C. 30 sec) using hSTC2 1U primer (Bioneer, Korea) and hSTC2 2D primer, obtaining 169 bp exon 1 fragment. According to the method as described above, 163 bp exon 2, 231 bp exon 3, and 420 exon 4 were obtained using hSTC2 3U primer and hSTC2 4D primer, hSTC2 5U primer and hSTC2 6D primer, and hSTC2 7U primer and hSTC2 8D primer, respectively.

Second PCR utilized exon 1 (169 bp), exon 2 (163 bp), exon 3 (231 bp) and exon 4 (420 bp) obtained by the above-described method as a template, and PCR using hSTC2 1U containing EcoRI (Takara, Japan) restriction site (GAATTC) and hSTC2 8D containing KpnI restriction site (GGTACC) was carried out for 30 cycles of 98° C. 10 sec; 55° C. 5 min; and 72° C. 1 min to obtain 926 bp stanniocalcin 2.

The resulting DNA encoding stanniocalcin 2 and pUC-18 (Amersham Pharmacia Biotech, Swiss) was restricted with EcoRI and KpnI (Takara, Japan), and ligated with T4 DNA ligase (Takara, Japan), followed by transformation into Top10F′ E coli. After incubating at 37° C. for 15 hrs, three colonies randomly selected were cultured and plasmids were obtained according to alkaline lysis method. These plasmids were electrophoresized on 1% agarose gel and then desirable plasmid (pUC-hSTC2) was selected by analysis using nucleotide sequence kit (Solgent, Korea).

PCR was carried out to link Met-stanniocalcin 2 in which narK promoter and signal sequence are removed, and primers were designed for base pairing between 18 bases in a linking region. The first PCR method is as follows: (a) pNKmut plasmid (−10 mutated narK promoter; Regeron Inc.) was commonly used as a template; and (b) PCR cycle (98° C. 10 sec; 55° C. 5 sec; and 72° C. 25 sec) using OY-17 and r-narK D primer pair, obtaining 350 bp narK promoter. PCR (30 cycles: 98° C. 10 sec; 55° C. 5 sec; and 72° C. 55 sec) was carried out using pUC-hSTC2 as a template and hSTC2 9U and hSTC2 8D primer pair to obtain 863 bp Met-stanniocalcin 2.

Second PCR utilized narK promoter (350 bp) and Met-stanniocalcin 2 (420 bp) obtained by the above-described method as a template, and PCR using OY-17 containing EcoRI (Takara, Japan) restriction site (GAATTC) and hSTC2 8D containing KpnI restriction site (GGTACC) was carried out for 30 cycles of 98° C. 10 sec; 55° C. 5 min; and 72° C. 1 min to obtain 1,195 bp fragments containing Met-stanniocalcin 2 which narK promoter and signal sequence is removed. 1,195 bp fragments (containing Met-stanniocalcin 2 which narK promoter and signal sequence is removed) and pUC-rrnB (rrnB terminator is inserted into pUC18; Regeron Inc.) were restricted with EcoRI and KpnI, and ligated with T4 DNA ligase, followed by transformation into Top10F′ E coil After incubating at 37° C. for 15 hrs, three colonies randomly selected were cultured and plasmids were obtained according to alkaline lysis method. These plasmids were electrophoresized on 1% agarose gel and then desirable plasmid (pUC-narK Met-hSTC2) was selected by analysis using nucleotide sequence kit.

TABLE
Primer nucleotide sequences.

| Primer | Nucelotide sequence (5′→3′) |
|---|---|
| hSTC2 1U | CCGGAATTCATGTGTGCCGAGCGGC (25mer) |
| hSTC2 2D | GGATCTCCGCTGTATTCTGCAGGGACAGG (29mer) |
| hSTC2 3U | CAGAATACAGCGGAGATCCAGCACTGTT (28mer) |
| hSTC2 4D | ATGACTTGCCCTGGGCATCAAATTTTCC (28mer) |
| hSTC2 5U | GATGCCCAGGGCAAGTCATTCATCAAAGAC (30mer) |
| hSTC2 6D | CACGTAGGGTTCGTGCAGCAGCAAGTC (27mer) |
| hSTC2 7U | GCTGCACGAACCCTACGTGGACCTCGT (27mer) |
| hSTC2 8D | GGGGTACCTCACCTCCGGATATCAGAATAC (30mer) |
| hSTC2 9U | GTATCAGAGGTGTCTATGACCGACGCCACCAACC |
| | (34mer) |
| OY-17 | CCGGAATTCGTAAACCTCTTCCTTCAGGCT (30mer) |
| r-narK D | CATAGACACCTCTGATACTCGTTTCG (26mer)] |

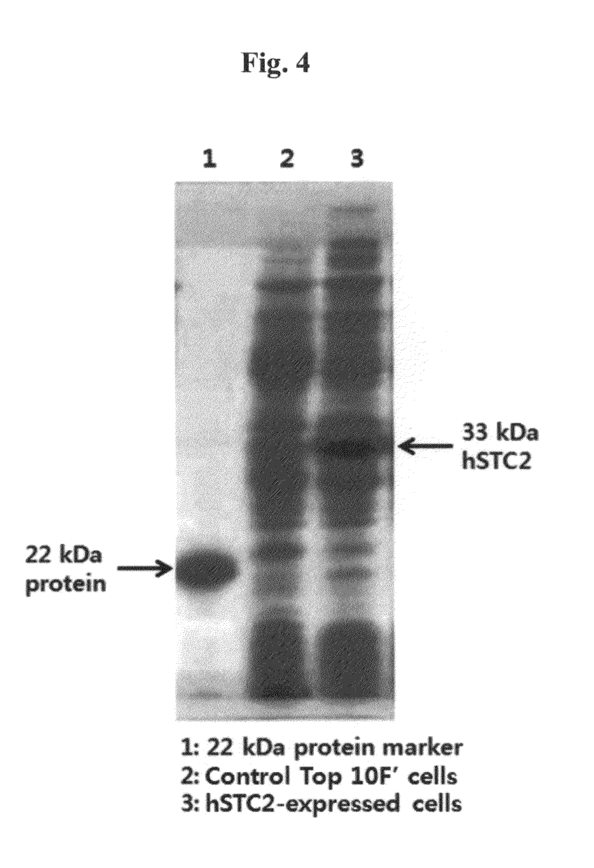

Ten g of Top10F′ cells transformed with pUC-narK Met-hSTC2 was suspended in 200 ml of 50 mM EDTA solution, and then sonicated, followed by centrifuging at 10,000 g for 30 min to collect precipitates. The precipitates were resuspended and then analyzed on SDS-PAGE. As shown in FIG. 4, about 33 kDa band indicating hSTC2 was observed. In addition, 33 kDa band on SDS-PAGE was eluted and incubated with trypsin (Promega, US) at 37° C. for 16 hrs. As a result, it could be demonstrated that the band is hSTC2 using MALDI-TOF (Applied Biosystems, US) and MS-Fit search (Protein Prospector).

The centrifuged precipitates were mixed to 200 ml distilled water, and 1 ml of 100% Triton X-100 was added to a concentration of 0.5%, followed by shaking at room temperature for 30 min. The precipitates were harvested by centrifuging at 10,000 g for 30 min. The precipitates were dissolved in 200 ml distilled water, and stirred at room temperature for 30 min. The precipitates were collected by centrifuging at 10,000 g for 30 min. After the precipitates were mixed with solution A (50 mM Tris pH 8.0, 6 M Urea, 10 mM 2-Mercaptoethanol), the mixture was stirred at room temperature for 90 min, and centrifuged at 10,000 g for 40 min, obtaining the supernatant.

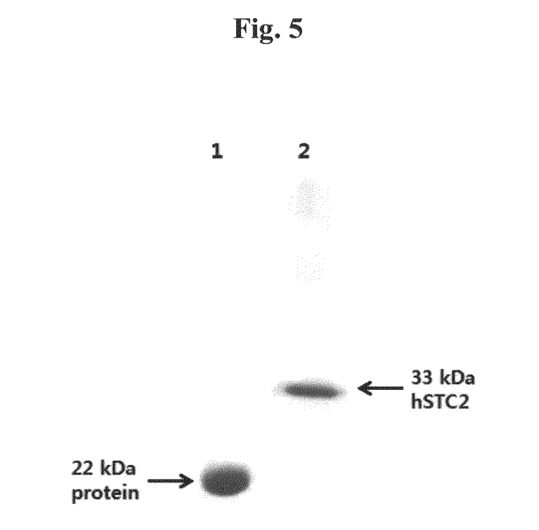

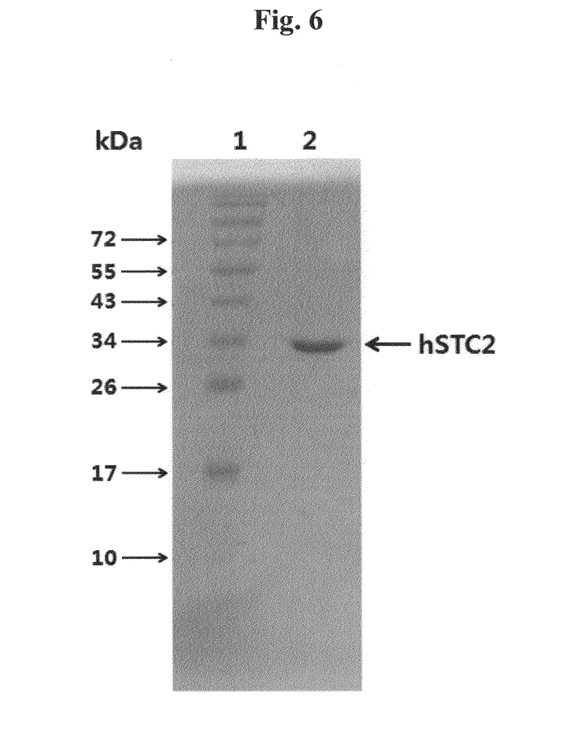

The supernatant was diluted with 200 ml distilled water, and adsorbed to gel by passing DEAE-Sepharose column (GE Healthcare) pre-equilibrated with a buffer solution (20 mM Tris, 1 mM EDTA), followed by washing with the buffer solution (20 mM Tris, 1 mM EDTA). The adsorbed proteins were eluted from the gel using a buffer solution (20 mM Tris, 1 mM EDTA, 300 mM NaCl). The eluent is subjected to gel filtration chromatography using Superdex 200 (GE Healthcare) pre-equilibrated with a buffer solution (20 mM NaH₂PO₄, 1 mM EDTA, pH 7.0). The eluted fractions were electrophoresized on 15% SDS-PAGE (FIG. 5) to collect only the fractions which hSTC2 purity is higher than 90%. Finally, purified hSTC2 (not less than 90% purity; FIG. 6) was measured at 595 nm using Bradford assay with standard protein (BSA; bovine serum albumin) and Spectra MAX 190 (Molecular Device Inc.), obtaining quantitative protein amount of 0.125 mg/ml. Final purified hSTC2 was utilized in further experiments.

### 2. Determination of Stanniocalcin 2 (STC2) Concentration for Intracerebroventricular (LCV) Injection

Five μl hSTC2 (100 ng/5 μl in PBS) was intracerebroventricularly injected to ICR mouse (DBL, Korea).

### 3. Intraperitoneal Injection of Bromodeoxyuridine (BrdU)

Fifty mg/kg BrdU was intraperitoneally injected to four-week-old male ICR mouse (DBL, Korea) with weight of 23-25 g.

### 4. Bromodeoxyuridine (BrdU) Staining

hSTC2 was intracerebroventricularly injected to four-week-old male ICR mouse (DBL, Korea) with weight of 23-25 g and then BrdU was intraperitoneally injected. After injection for 24 hrs, experimental animals were subjected to perfusion fixation using 4% paraformaldehyde preperfusion solution. Afterward, brain was immediately extracted from the animals and was washed with 30% sucrose solution for 24 hrs after postfixation in equal solution for 4 hrs. The brain tissues were frozen using optimum cutting temperature compound (OCT compound, Fisher). Tissue sections with 40 μm thickness was prepared using a freezing microtome and added with cryoprotectant solution, followed by being stored at −20° C. for BrdU staining. Experiments were carried out for 2 days. In the first day, brain tissues immersed in cryoprotectant solution were transferred to acryl plate well, and washed three times with 50 mM phosphate buffer (PB) for 5 min. After treatment with 0.5% Triton X-100 for 20 min, the tissues were washed three times with 50 mM phosphate buffer (PB) for 5 min, and transferred into glass bottle containing 2 ml solution which is composed of 50% formamide and 2×SSC prepared using 100% formamide and 4×SSC, followed by incubating at 65° C. for 2 hrs in a shaking incubator. After washing with 2×SSC two times for 5 min, the tissues were added with 2 N HCl (9.6 ml PBS+2 ml HCl conc.) prewarmed at 37° C. for 30 min in a shaking incubation bath, and neutralized at 25° C. for 10 min in 0.1 M sodium borate (pH 8.5) with shaking. The tissues were washed three times with 50 mM PB for 5 min, and incubated with 1% BSA (bovine serum albumin) and 10% horse serum for 1 hr, followed by immunohistochemical staining at 4° C. for 12 hrs using anti-BrdU antibody (Roche). Next day, the brain tissues were washed three times with 50 mM PB for 5 min, and incubated with biotin-conjugated goat anti-mouse IgG secondary antibody (1:200, Vector) contained in 50 mM PB and 0.5% BSA for 1 hr, followed by washing three times with 50 mM PB for 5 min. The tissues were incubated with ABC (avidin-biotin complex) reagent (1:200, Vector) for 1 hr, and washed three times with 50 mM PB for 5 min, followed by colorimetric reaction using diaminobenzidine (DAB) as a substrate. After stopping reaction, the tissues were stained with cresyl violet for about 2 min, and had transparent by dehydration using conventional methods. Finally, the tissues were embedded in polymount.

### 5. Immunohistochemistry

Five μl of kainic acid (0.1 μg/5 μl, Tocoris), or 5 μl of mixture solution (containing 0.1 μg kainic acid and 100 ng hSTC2 in 5 μl solution) was intracerebroventricularly (I.C.V) injected to four-week-old male mouse with weight of 23-25 g. After injection for 24 hrs, experimental animals were subjected to perfusion fixation using 4% paraformaldehyde preperfusion solution. Afterward, brain was immediately extracted from the animals and was washed with 30% sucrose solution for 24 hrs after postfixation in equal solution for 4 hrs. The brain tissues were frozen using OCT compounds. Tissue sections with 40 μm thickness was prepared using a freezing microtome and added with cryoprotectant solution, followed by being stored at −20° C. for immunohistochemistry. In first experimental day, brain tissues immersed in cryoprotectant solution were washed three times with 50 mM PB for 5 min. The tissues was treated with 3% H₂O₂ (in 50 mM PB) for 10 min to remove endogenous peroxidase, and incubated with 50 mM PB, 1% BSA and 0.2% Triton X-100 for 30 min. After incubating with 50 mM PB, 0.5% BSA and 3% normal serum for 1 hr, the tissues were washed with 50 mM PB for 10 min, and immunohistochemically stained with using anti-OX-42 monoclonal antibody. Next day, the brain tissues were washed three times with 50 mM PB for 5 min, and incubated with goat anti-mouse IgG secondary antibody (1:200) contained in 50 mM PB and 0.5% BSA for 1 hr, followed by washing three times with 50 mM PB for 5 min. The tissues were incubated with ABC reagent (1:200) for 1 hr, and washed three times with 50 mM PB for 5 min, followed by colorimetric reaction using DAB as a substrate. After stopping reaction, the tissues had transparent by dehydration using conventional methods and finally embedded in polymount.

### 6. BV2 Microglia Culture

Mouse microglia cell line, BV2 (kindly provided by Dr. Cho Dong-Hyup, Department of Neurobiology and Behavior, Cornell University), was cultured in DMEM (Dulbeco's Modified Eagle's Medium) media (GIBCO BRL, USA) supplemented with 2 mM L-glutamine, 100 U/ml penicillin, 100 μg/ml streptomycin, 10% heat-inactivated fetal bovine serum (FBS) at 37° C. in 5% CO₂ incubator. Cells were subcultured when they were grown to about 90% of bottom area, and cells of exponential growth phase were used for further experiments.

### 7. Drug Treatment

To inhibit microglia activity, hSTC2 was treated at a final concentration of 10 nM. As a microglia activator, LPS (lipopolysaccharide) was treated at a final concentration of 200 ng/ml.

### 8. Measurement of Nitric Oxide (NO) Concentration

The production of nitric oxide (NO) was determined by measuring concentration of nitrite (NO₂⁺). The concentration of nitrite was measured by colorimetric assay using Griess reagent (1% sulfanilamide, 0.1% naphthyl-ethylenediamine dihydrochloride/2.5% H₃PO₄).

### 9, Mouse Y-Maze Test

Four-week-old male ICR mouse (DBL, Korea) with weight of 23-25 g was randomly divided into two groups (control and test), and each group consists of five mice. Five μl of kainic acid (0.1 μg/5 μl, Tocoris) and 5 μl of mixture solution (containing 0.1 μg kainic acid and 100 ng hSTC2 in 5 μl solution) were intracerebroventricularly (I.C.V) injected to control and test group, respectively. After injection for 24 hrs, Y-maze experiment was performed to examine cognitive function. Y-maze device is composed of three arms with 40 (width)×12 (length)×30 (height), and experiment was carried out in intensity of illumination of 20±5 lux. Each three arms consisting of Y-maze was randomly named as A, B and C. After a head part of mouse was put toward the passage in the end of an arm, mouse wandered into the passage in a free manner for 8 min to observe movement path. Passing of the arm on Y-maze of this invention means that hind legs of mouse are entered into the passage of an arm. As described above, arms that mouse passes were sequentially recorded and then tied up three in a sequence. As a result, it was considered as one point that all paths (arms) is independently different, which mouse passes. For example, where mouse passes the arm in a sequence of ABCAC, the order of ABC, BCA and CAC is tied, giving two points. Memory score (%) is calculated as follows: total score is divided by (total path number-2) and converted to percentage.

### 10. Mouse Water Finding Test

Four-week-old male ICR mouse (DBL, Korea) with weight of 23-25 g was randomly divided into two groups (control and test), and each group consists of five mice. Five μl of kainic acid (0.1 μg/5 μl, Tocoris) and 5 μl of mixture solution (containing 0.1 μg kainic acid and 100 ng hSTC2 in 5 μl solution) were intracerebroventricularly (I.C.V) injected to control and test group, respectively. After injection for 24 hrs, water finding test was performed to suppose latent learning. A device was a box in a size of 30 (width)×50 (length)×20 (height), and its bottom was divided into 15 spaces of 10×10 cm, of which a door of 10×10 cm was prepared one wall, and a water bottle was put inside the door. In first day, mouse injected with kainic acid alone or kainic acid and STC2 was placed in one end of the space and learned to drink water. After learning, the supply of water was stopped for 24 hrs. In the second day, the mouse was again put into the device, and then the time (sec) of drinking latency was measured.

### 11. Mouse Forced Swim Test

Four-week-old male ICR mouse (DBL, Korea) with weight of 23-25 g was randomly divided into two groups (control and test), and each group consists of five mice. Five μl of PBS and 5 μl of hSTC2 (100 ng/5 μl) were intracerebroventricularly (I.C.V) injected to control and test group, respectively. After injection for 24 hrs, immobilization stress was forced for 2 hrs. After stress, the mouse was subjected to forced swim for 6 min in circular water bath (diameter, 10 cm; height, 20 cm) containing water of 25±2° C. Two min later, the time in an immobile floating posture that the face of mouse is floated on the surface of the water was measured for 4 min. An immobile behavior is known to be helplessness.

### 12. Effects on Brain Impair Caused by Transient Focal Cerebral Ischemia and Middle Cerebral Artery Occlusion (MCAO)

Ten of adult male C57BL/6J mice (3-month-old, 25-30 g, DBL, Korea) were anesthetized by intraperitoneal injection with tiletamine, zoletile and xylazine hydrochloride (8 mg/kg), and immobilized on stereotaxic instrument (Harvard Apparatus). After the skin was dissected on centerline, brain injector (Harvard Apparatus) was inserted with a depth of 2.5 mm into a position of 0.2 mm and 1.2 mm in the back and lateral direction of bregma, respectively. The brain injector was immobilized by dental cements. Three day later, mouse was anesthetized with face mask using 2% isoflurane (Tocoris) and gas mixture (70%/30%) of nitrogen and oxygen, and body temperature was maintained at 37±0.5° C. using heating pad and lamp. Mice were randomly divided into two groups (control and test), and each group consists of five mice. Using brain injector, Five μl of PBS and 5 μl of hSTC2 (100 ng/5 μl) were intracerebroventricularly (I.C.V) injected to control and test group, respectively. Midline cervical cleft was incised to expose external carotid artery, and 5.0 nylon suture (Ethicon, Edinburg, UK) in a length of 9.0 mm, of which the tip was blunt with heat treatment was inserted into internal carotid artery through external carotid artery, blocking blood flow to middle cerebral artery. After 60 min, blood flow was recovered by removal of nylon. 24 hrs after focal cerebral ischemia, mice were sacrificed and their brains were extracted. For coronal section, the brain tissues were cut from frontal lobe in a thickness of 1 mm using a brain matrice (Harvard Apparatus). Each fragment was incubated in 2% TTC (2,3,5-triphenyltetrazolium chloride) at 37° C. for 15 min, and stained. Through scanning with a scanner (1,200 dpi; Hewlett-Packard), the images were analyzed using ImagePro-Plus software (Media Cybernetics).

### Results

Effect of Stanniocalcin 2 (STC2) on Kainic acid(KA)-Inducible Neuronal Death

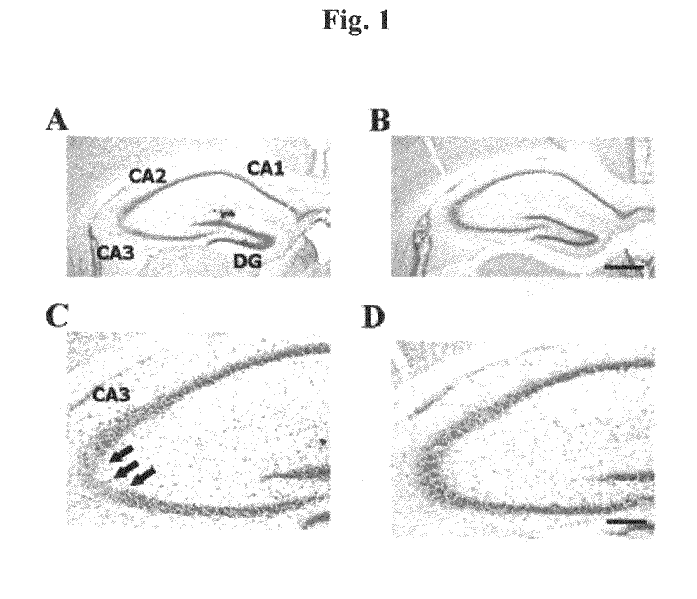

In this study, the present inventors examined whether pyramidal neuronal death (practically, apoptosis of neuronal cells) in hippocampal CA3 region by KA is inhibited by STC2. Five μl of mixture solution (containing 0.1 μg kainic acid and 100 ng hSTC2 in 5 μl solution) was intracerebroventricularly (I.C.V) injected to male ICR mouse with weight of 23-25 g. 24 hrs after injection, the brain tissues were extracted. The brain tissue sections were stained with cresyl violet to observe neuronal death in hippocampal CA3 region. As a result, it was demonstrated that pyramidal neuronal death in hippocampal CA3 region was produced in one group treated with KA alone, whereas inhibited in the other group treated with both KA and STC2 (FIG. 1). According to the present invention, it could be appreciated that STC2 plays a protective role in excessive neurotoxicity.

### Effects of Stanniocalcin 2 (STC2) on Neurogenesis

It has been known as neurogenesis that neuron in a part of brain is proliferated and differentiated although differentiation of neuronal cell is finished. Neurogenesis is generated in subgranular zone (SGZ) beneath granular cell layer (GCL) of dentate gyrus (DG) in hippocampus which is responsible for memory and cognitive function in brain, and is known to be promoted by learning.

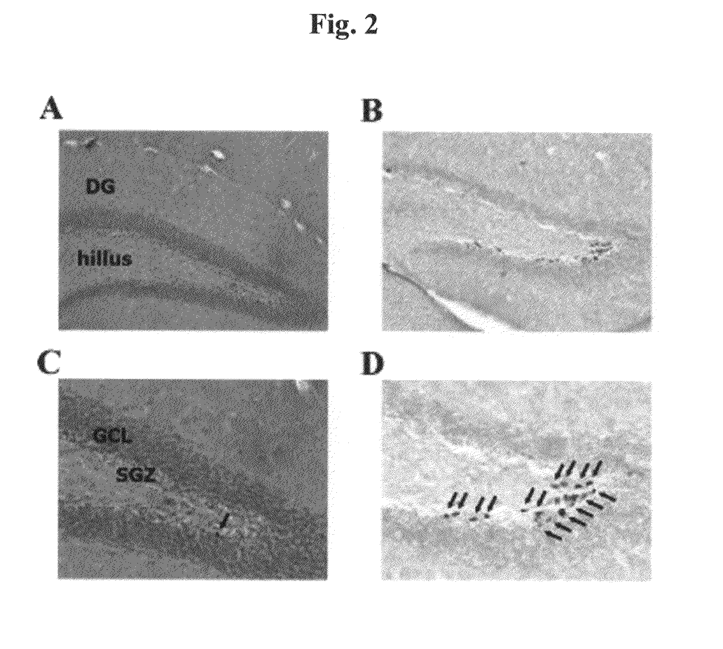

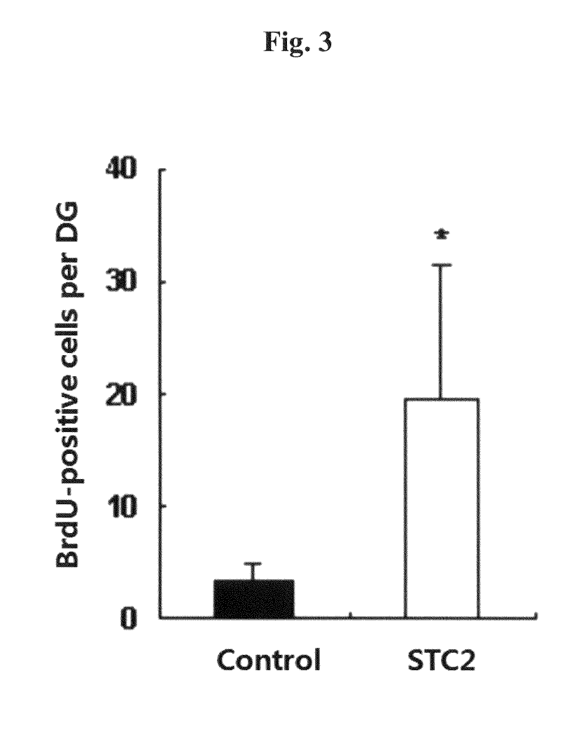

STC2 (10 nM) was intracerebroventricularly (I.C.V) injected to male ICR mouse with weight of 23-25 g, and bromodeoxyuridine (BrdU; 100 mg/kg) was intraperitoneally injected to male ICR mouse with weight of 23-25 g. 24 hrs after injection, the brain tissues were extracted and subjected to BrdU immunohistochemistry. As a result, it could be demonstrated that BrdU-immunopositive cells in a group treated with STC2 are increased in SGZ of hippocampus compared to control (FIG. 2 and FIG. 3). According to the present invention, it could be appreciated that STC2 promotes neurogenesis.

Effects of Stanniocalcin 2 on Y-Maze Test in Mouse Treated with Kainic Acid

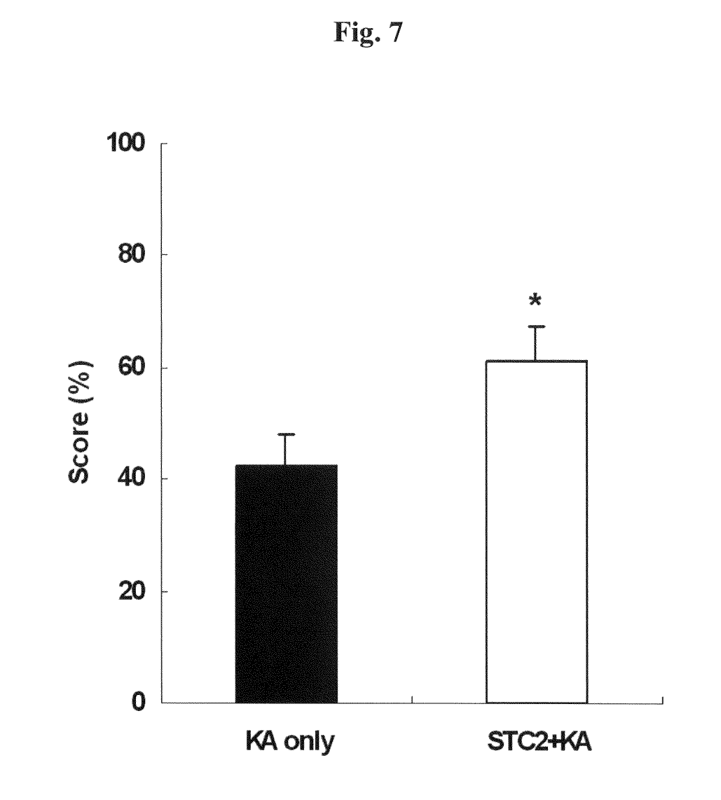

The present experiment is carried out to examine place memory function using research and curiosity which are basic characteristics in rodents. Memory score was 42.5±5.4% in KA (kainic acid) alone-treated group and 61.3±6.3% in the group treated with both hSTC2 and KA, suggesting that memory score decreased by KA is remarkably enhanced by hSTC2 (FIG. 7).

Effects of Stanniocalcin 2 on Water Finding Test in Mouse Treated with Kainic Acid

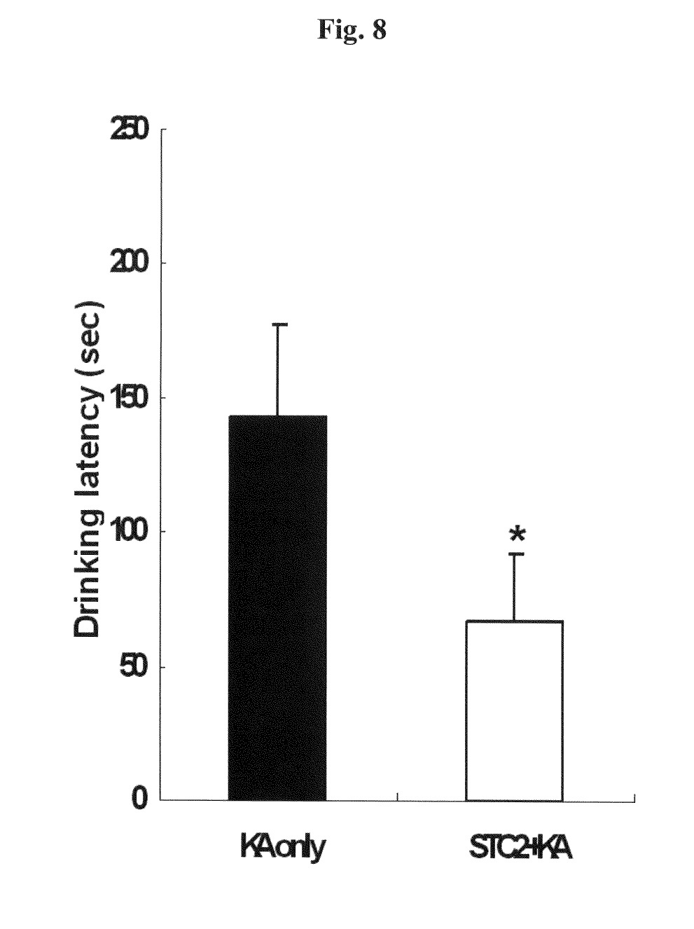

The present experiment is carried out to estimate learning, place memory and working memory. In high level of learning and memory function, drinking latency is relatively short. Drinking latency in hSTC2-treated group (67±25 sec) is significantly decreased compared to that in KA alone-treated group (143±34 sec) (p<0.05) (FIG. 8).

Effects of Stanniocalcin 2 on Forced Swim Test in Mouse Treated with Kainic Acid

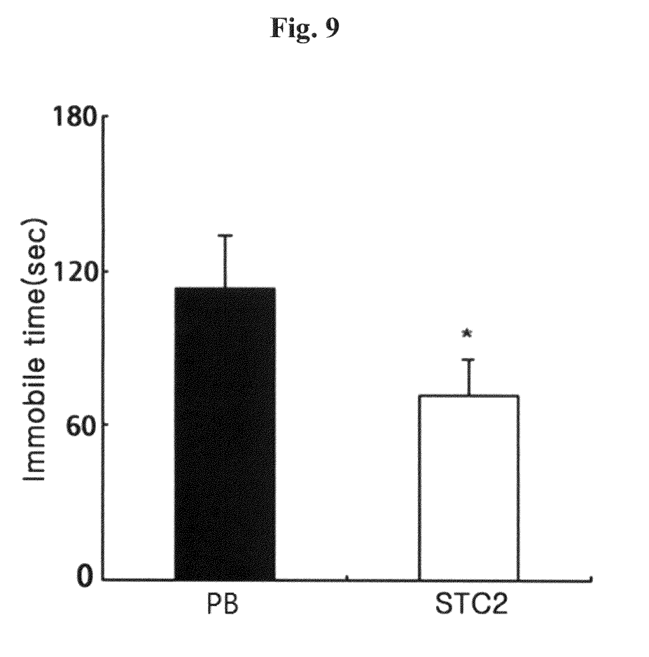

The present test is commonly utilized as a depression animal model for observation and assessment of depression-related behavior. The longer immobile time criterion, the higher helplessness estimated from the test. Immobile time in hSTC2-treated group (71±15 sec) is significantly reduced compared to that in a KA alone-treated group (113±21 sec) (p<0.05) (FIG. 9).

### Effects of Stanniocalcin 2 on Brain Impair Caused by Transient Focal Cerebral Ischemia and Middle Cerebral Artery Occlusion (MCAO)

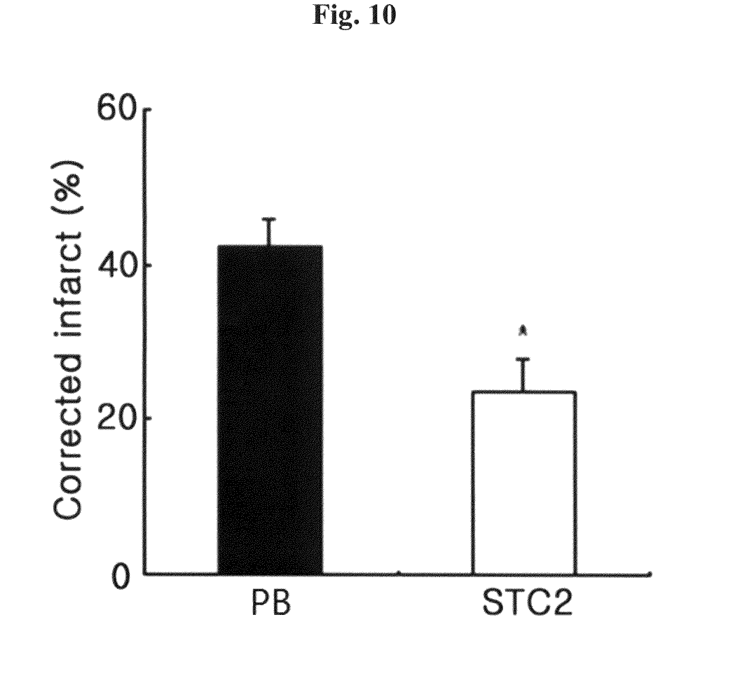

The present experiment is carried out to determine whether injection of hSTC2 decreases cerebral infraction and neurological deficit. The size of cerebral infraction in hSTC2-treated group (23.8±4.2 sec) is significantly reduced compared to that in KA alone-treated group (42.2±3.4 sec) (p<0.05) (FIG. 10).

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A method for preventing or treating a brain disease, which comprises administering to a subject a pharmaceutical composition comprising stanniocalcin 2 as an active ingredient.

2. A method for improving a cognitive function, which comprises administering to a subject a pharmaceutical composition comprising stanniocalcin 2 as an active ingredient.

3. The method according to claim 1, wherein the composition is a pharmaceutical composition or a food composition.

4. The method according to claim 1, wherein the composition has a protective activity for neuronal cells.

5. The method according to claim 4, wherein the protective activity for neuronal cells is exhibited by inhibiting a neuronal apoptosis.

6. The method according to claim 1, wherein the composition has an activity for promoting a neurogenesis.

7. The method according to claim 1, wherein the brain disease is selected from the group consisting of a neurodegenerative disease, an ischemia-reperfusion injury or a mental disorder.

8. The method according to claim 7, wherein the mental disorder is depression or manic depression.

9. The method according to claim 2, wherein the cognitive function is learning ability, memory or concentration.


SEQUENCE LISTING (11 sequences)

SEQ ID NO: 1 - DNA, 25 bp, Homo sapiens
ccggaattcatgtgtgccgagcggc

SEQ ID NO: 2 - DNA, 29 bp, Homo sapiens
ggatctccgctgtattctgcagggacagg

SEQ ID NO: 3 - DNA, 28 bp, Homo sapiens
cagaatacagcggagatccagcactgtt

SEQ ID NO: 4 - DNA, 28 bp, Homo sapiens
atgacttgccctgggcatcaaattttcc

SEQ ID NO: 5 - DNA, 30 bp, Homo sapiens
gatgcccagggcaagtcattcatcaaagac

SEQ ID NO: 6 - DNA, 27 bp, Homo sapiens
cacgtagggttcgtgcagcagcaagtc

SEQ ID NO: 7 - DNA, 27 bp, Homo sapiens
gctgcacgaaccctacgtggacctcgt

SEQ ID NO: 8 - DNA, 30 bp, Homo sapiens
ggggtacctcacctccggatatcagaatac

SEQ ID NO: 9 - DNA, 34 bp, Homo sapiens
gtatcagaggtgtctatgaccgacgccaccaacc

SEQ ID NO: 10 - DNA, 30 bp, Homo sapiens
ccggaattcgtaaacctcttccttcaggct

SEQ ID NO: 11 - DNA, 26 bp, Homo sapiens
catagacacctctgatactcgtttcg